(19) United States
(12) Patent Application Publication
(10) Pub. No.: US 2006/0134174 A1
(43) Pub. Date: Jun. 22, 2006

(54) Pharmaceutical delivery system and method of use

(75) Inventors: Stephen Bartels, Pittsford, NY (US); Dharmendra Jani, Fairport, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/313,856

(22) Filed: Dec. 21, 2005

Related U.S. Application Data
(60) Provisional application No. 60/638,764, filed on Dec. 22, 2004.

(51) Int. Cl. A61K 31/407; A61F 2/00

(52) U.S. Cl. 424/427; 514/410

(57) ABSTRACT

The present invention includes a pharmaceutical delivery system comprising a fused pyrrolocarbazole and a biodegradable polymer matrix configured to be inserted into the eye of the patient.

### CROSS REFERENCE

This application claims the benefit of Provisional Patent Application No. 60/638,764 filed Dec. 22, 2004 and is incorporated herein by reference.

### BACKGROUND OF THE INVENTION

1. Field of the Invention

The present invention relates generally to pharmaceutical delivery systems, pharmaceutical compositions, methods of use thereof and methods of manufacture thereof for treatment of disease regulated by tyrosine kinase in the ocular region of a patient. More particularly, the present invention relates to pharmaceutical delivery systems, pharmaceutical compositions, methods of use thereof and methods of manufacture thereof for delivering VEGF receptor inhibitors to the ocular region of a patient.

2. Discussion of Related Art

For many years it has been known that treatment of eye disease with a pharmaceutical agent presented challenges because the eye has natural membrane barriers that prevent passage of the pharmaceutical agent into the ocular region. These barriers include the blood-retinal barrier, the cornea, etc. Consequently, systemic treatment of tissue in the eye often requires the level of pharmaceutical agents in the blood plasma to be relatively higher than the therapeutic levels of the pharmaceutical agent in the tissues of the eye to achieve an efficacious result. Application of a pharmaceutical agent topically to the eye also requires passing the pharmaceutical agent through the membrane barriers of the eye such as the cornea. Pharmaceutical agents can be administered to the tissue inside the eye of a patient by a bolus injection. Patients generally dislike the use of bolus injections because of its invasive nature.

Pharmaceutical delivery devices and compositions (i.e. pharmaceutical delivery systems) are currently under development to deliver pharmaceutical agents to the eye of a patient. While placement of a pharmaceutical delivery system is possibly more invasive than a bolus injection, patients expect a pharmaceutical delivery system to deliver the medicament for a longer period of time reducing the requirement for multiple repeated injections into the eye of the patient. Nonetheless, extended release pharmaceutical delivery systems are new, and few medicines can be delivered to the interior portion of the eye by techniques other than a bolus injection.

Examples of extended release pharmaceutical delivery systems are found in US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); U.S. Pat. No. 6,331,313 (Wong et al); and U.S. patent application Ser. No. 10/610,063 (Drug Delivery Device, filed Jun. 30, 2003) (Mosack) all of, which are incorporated by reference. Publications cited throughout this disclosure are incorporated in their entirety herein by reference.

Additionally, US Patent Application Publication 2003/0095995 discloses a formulation for controlled release of drugs by combining hydrophilic and hydrophobic agents. A biodegradable matrix, including polylactate-polyglycolate, is mixed with one or more pharmaceutical agents including corticosteroids and a release modifier. The biodegradable polymer matrix is injected into the eye of a patient and delivers the pharmaceutical agent to the surrounding tissue.

It has been known for some time that tyrosine kinase inhibitors can be used potentially to treat eye disease. U.S. Pat. Nos. 5,980,929 and 5,919,813 and WO Publication No. 2000/67,738 discloses the use of genistein as a protein tyrosine kinase pathway inhibitor in the treatment of retinal ischemia, diabetic retinopathy, ocular inflammation, age-related macular degeneration and other ocular disorders. Each of these patents discuss administration by injection in addition to other systemic forms of administration.

Various synthetic small organic molecules that are biologically active and generally known in the art as “fused pyrrolocarbazoles” have been prepared. Examples of such patents include U.S. Pat. Nos. 5,475,110, 5,591,855, 5,594,009, 5,616,724 and 5,705,511. The fused pyrrolocarbazoles were disclosed to be used in a variety of ways, including inhibition of protein kinase C (“PKC”), inhibition of trk tyrosine kinase activity and inhibition of the cellular pathways involved in the inflammation process.

Certain selected fused pyrrolocarbazoles are taught in U.S. Pat. No. 6,630,500 to have activity for inhibition of VEGFR2 as a potential therapeutic for treatment of ocular disease such as retinopathy (including diabetic retinopathy), edema (including macular edema) and ocular inflammation.

U.S. Application Publication No. U.S. 2004/0167091 discloses a biodegradable pharmaceutical delivery system for delivery of anti-VEGF therapy that combines an agent that inhibits the development of neovascularization and particularly an oligonucleotide, with a biodegradable matrix material selected from the group consisting of lactide polymers, lactide/glycolide copolymers, or polyoxyethylene-polyoxypropylene copolymers.

Nonetheless, there is still a need for a drug-delivery system that can be inserted into the eye to deliver a pharmaceutical agent including a tyrosine kinase pathway inhibitor. The present invention addresses these and other needs.

### SUMMARY OF THE INVENTION

The present invention is a pharmaceutical delivery system comprising a fused pyrrolocarbazole and a biodegradable polymer matrix that is sized and configured to be inserted into the eye of the patient. It has been discovered that the delivery of a fused pyrrolocarbazole with a polymer matrix material provides sustained prolonged exposure to levels of dosing while avoiding repeated exposure to higher initial concentrations found after a bolus injection. The pharmaceutical delivery system controls the amount of fused pyrrolocarbazole in the patient's eye and potentially reduces or eliminates side effects from a bolus injection. In another embodiment, there is a method for treating angiogenic disorders in the eye of a patient, which comprises administering to a host in need of such treatment a pharmaceutical delivery system comprising a biodegradable polymer matrix and a therapeutically effective amount of a fused pyrrolocarbazole.

In one embodiment, the fused pyrrolocarbazole is selected from the group consisting of an indolocarbazole and an indenocarbazole and mixtures thereof.

In another embodiment, the fused pyrrolocarbazole is a compound defined by the following formula and salts thereof and prodrugs thereof and mixtures of the compound, salt and prodrug thereof: 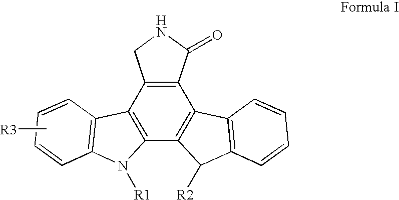
wherein:

R1 and R2 are the same or different and are independently selected from —H, or alkyl of 1-8 carbons, preferably an alkyl of 1-4 carbons, substituted with —OH, or —OR4 where R4 is an alkyl of 1-4 carbons, aryl, preferably phenyl or naphthyl, or the residue of an amino acid after the hydroxyl group of the carboxyl group is removed; and

R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6; and wherein

R5 is alkyl of 1-4 carbons or aryl, preferably phenyl or naphthyl;

R6 is H, alkyl of 1-4 carbons, aryl of 6-10 carbons, preferably phenyl or naphthyl, or heteroaryl;

R7 is H or alkyl of 1-4 carbons;

R8 is the residue of an amino acid after the hydroxyl group of the carboxyl group is removed;

n is an integer of 1-4; and

y is 1 or 2.

In still another embodiment, the fused pyrrolocarbazole is one or more compounds defined by Formula II and salts thereof and prodrugs thereof and mixtures of the compounds, salts and prodrugs thereof: 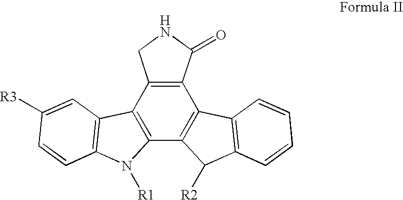
R₁ and R₂ are the same or different and are independently selected from —H, or alkyl of 1-8 carbons, substituted with —H, —OH or —OR4 where R4 is an alkyl of 1-4 carbons, aryl or the residue of an amino acid after the hydroxyl group of the carboxyl group is removed; and R3 is —CH₂OH; —CH₂OR7; —(CH₂)ₙSR5; —(CH₂)ₙSOₘR5; —CH₂SR₅; or alkyl of 1-8 carbons substituted with —OH, —OR5, —OR8, —CH₂OR7, —S(O)ₘR6 or —SR6; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and m is 1 or 2.

In one embodiment, the fused pyrrolocarbazole is defined according to Formula I or Formula II and R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)ₘR6 or —SR8; and wherein R5 is alkyl of 1-4 carbons or aryl; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

In another embodiment, there is a fused pyrrolocarbazole as defined in Formula I or Formula II wherein R1 is —CH₂CH₂CH₂OH or —CH₂CH₂CH₂OCOCH₂N(CH₃)₂; R2 is H; and R3 is —CH₂OR7 wherein R7 is alkyl of 1-4 carbons.

In still another embodiment, there is a fused pyrrolocarbazole as defined in Formula I or Formula II consisting of compounds represented in Table I (listed below) and salts thereof and prodrugs thereof and mixtures of the salts and prodrugs thereof:

TABLE 1

| | |
|---|---|
| | Formula I |
| 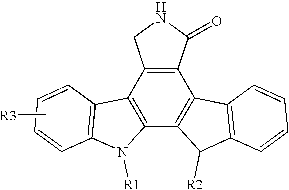 | |

| CMPD | | | |
|---|---|---|---|
| NO | R1 | R2 | R3 |
| 1 | —CH₂CH₂CH₂OH | —H | —CH₂OCH₃ |
| 2 | —CH₂CH₂CH₂OH | —H | —CH₂OCH(CH₃)₂ |
| 3 | —CH₂CH₂CH₂OH | —H | —CH₂O— |
| | | | CH(CH₃)CH₂CH₃ |
| 4 | —CH₂CH₂CH₂OH | —H | (S) —CH₂O— |
| | | | CH(CH₃)CH₂CH₃ |
| 5 | —CH₂CH₂CH₂OH | —H | (R) —CH₂O— |
| | | | CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂ |
| | | | CH₂CH₃ |
| 9 | —CH₂CH₂CH₂OH | —H | —CH(CH₃)OCH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —H | | —CH₂OCH₂CH₃ |
| 14 | —CH₂CH₂CH₂OH | —H | —CH(CH₃)O— |
| | | | CH₂CH₂CH₂CH₃ |
| 15 | —CH₂CH₂CH₂OH | —H | —CH(CH₃)O— |
| | | | CH(CH₃)₂ |
| 16 | —CH₂CH₂CH₂OH | —H | —CH₂OC(CH₃)₃ |
| 17 | —CH₂CH₂CH₂OCO— | —H | —CH₂OCH(CH₃)₂ |
| | CH₂NH₂ | | |
| 18 | —CH₂CH₂CH₂OCO— | —H | —CH₂OCH(CH₃)₂ |
| | CH₂NH₂— | | |
| | CH₂CH₂CH₂CH₂NH₂ | | |
| 19 | —CH₂CH₂CH₂OCOCH₂— | —H | —CH₂OCH(CH₃)₂ |
| | CH₂NH₂ | | |
| 20 | —CH₂CH₂CH₂OCOCH₂— | —H | —CH₂OCH(CH₃)₂ |
| | CH₂CH₂N(CH₃)₂ | | |
| 21 | —CH₂CH₂CH₂OCO— | —H | —CH₂OCH(CH₃)₂ |
| | CH₂N(CH₂)₂ | | |
| 22 | —CH₂CH₂CH₂OCO— | —H | —CH₂OCH(CH₃)₂ |
| | CH₂CH₂CH₂ | | |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−) |
| | | | —CH(OCH₃)CH₃ |
| 34 | —CH₂CH₂CH₂OH | —CH₂OH | —CH₂OCH(CH₃)₂ |

In another embodiment, the fused pyrrolocarbazole is of the following formula and salts thereof and prodrugs thereof and mixtures of the compound, salts and prodrugs thereof:

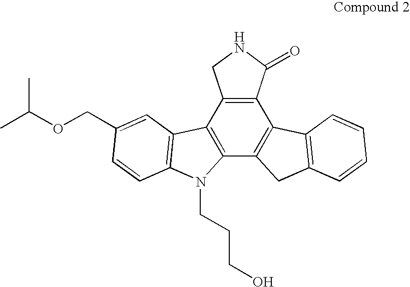

In still another embodiment, the fused pyrrolocarbazole is a compound of the following formula and salts thereof and prodrugs thereof and mixtures of the compound, salts and/or prodrugs thereof:

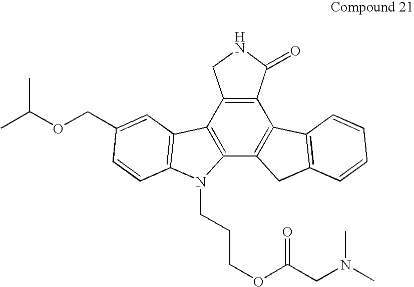

### DETAILED DESCRIPTION OF PREFERRED EMBODIMENTS

The present invention is a pharmaceutical delivery system comprising a fused pyrrolocarbazole and a biodegradable polymer device configured to be inserted into the eye of the patient. It has been discovered that the delivery of a fused pyrrolocarbazole with a pharmaceutical delivery system according to one or more embodiments of the present invention provides sustained prolonged exposure to levels of dosing while avoiding repeated exposure to higher initial concentrations found after a bolus injection. The pharmaceutical delivery system controls the amount of fused pyrrolocarbazole in the patient's eye and potentially reduces or eliminates side effects that may result from a bolus injection. In another embodiment, there is a method for treating angiogenic disorders in the eye of a patient, which comprises administering to a host in need of such treatment a pharmaceutical delivery system comprising a biodegradable polymer matrix and a therapeutically effective amount of a fused pyrrolocarbazole.

Definitions

“Pharmaceutically acceptable salts” is defined as a salt formed by addition of an acid to a base containing organic molecule or a base to an acid containing organic molecule.

“Fused pyrrolocarbazole” is defined as a compound having a fused pyrrolocarbazole core structure as shown in the following Formula IV: 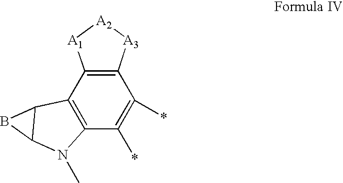
wherein at least one of A1, A2 or A3 is a nitrogen B is a structure that forms an aryl or heteroaryl ring systems with the carbon atoms to, which B is bonded. The designation * indicates the attachment point of an additional fused ring system.

The core structures provided herein are presented by way of the general guidance and are not to be taken as limiting the scope of the invention. For example, certain cores indicate the presence of certain atoms for illustrative purposes. It will be appreciated that such atoms may be bonded to additional groups, or may be further substituted without deviating from the spirit of the invention.

Thus, fused pyrrolocarbazole core structures include, but are not limited to, structures of formula V as follows:

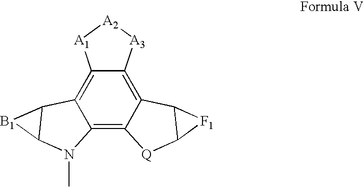

wherein at least one of A1, A2 and A3 is a nitrogen, B1 and F1 together with the adjacent carbons to, which they are attached independently form an aryl or heteroaryl ring. Q is a moiety containing one or more nitrogen atoms or carbon atoms. Such structures include but are not limited to indolocarbazoles, indenocarbazoles and bridged indenocarbazoles.

As used herein, “indolocarbazole” is intended to indicate a compound of formula V, wherein at least one of A1, A2 and A3 is a nitrogen. B1 and F1 together with the adjacent carbons to, which they are attached independently form an aryl or heteroaryl ring. Q is nitrogen.

As used herein, “indenocarbazole” is intended to indicate a compound of formula V, wherein at least one of A1, A2 and A3 is a nitrogen. B1 and F1 together with the adjacent carbons to, which they are attached independently form an aryl or heteroaryl ring. Q is a substituted or unsubstituted carbon atom.

“Inflammation-mediated condition of the eye” is defined as any condition of the eye, which may benefit from treatment with an anti-inflammatory agent and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic opthalmia, Vogt Koyanagi-Harada (VKH) syndrome, histoplasmosis and uveal effusion.

“Angiogenesis-mediated condition of the eye” is defined as any condition of the eye that is caused by the pathway for growth of new blood vessels. Some angiogenesis-mediated condition of the eye includes but are not limited to ocular neovascularization including neovascularization of the cornea, iris, retina, as well as choroidal neovascularization associated with histoplasmosis, pathological myopia, age-related macular degeneration, angioid streaks, anterior ischemic optic neuropathy, bacterial endocarditis, Best's disease, birdshot retinochoroidopathy, choroidal hemangioma, choroidal nevi, choroidal nonprofusion, choroidal osteomas, choroidal rupture, choroderemia, chronic retinal detachment, coloboma of the retina, drusen, endogenous Candida endophthalmitis, extrapapilary hamartoma of the retinal pigmented epithelium, fundus flavimaculatus, idiopathic macular hole, malignant melanoma, metallic intraocular foreign body, morning glory disc syndrome, multiple evanescent, white-dot syndrome, neovascularization at ora serrata, operating microscope burn, optic nerve head pits, photocoagulation, punctuate inner choroidopathy, radiation retinopathy, retinal cryoinjury, retinitis pigmentosa, retinochoroidal coloboma, rubella, sarcoidosis, serpiginous or geographic choroiditis, subretinal fluid drainage, tilted disc syndrome, Taxoplasma retinchoroiditis, tuberculosis or Vogt-Koyanagi-Harada syndrome.

The term “biodegradable polymer” is defined as polymers that degrade in vivo and wherein erosion of the polymer over time is required to achieve the agent release kinetics according to the invention. Specifically, hydrogels such as methylcellulose, which act to release drug through polymer swelling, are specifically excluded from the term “biodegradable polymer.”

The terms, “inhibit” and “inhibition” are defined as a specified response of a designated material (e.g., enzymatic activity) is comparatively decreased in the presence of a fused pyrrolocarbazole of the present invention.

The term “contacting” is defined as directly or indirectly causing placement together of two items, such that the two items directly or indirectly come into a physical or chemical association with each other to affect a particular outcome.

As used herein, “prodrug” is intended to include any covalently bonded carrier, which releases the active parent pharmaceutical agent as a compound of the present invention in vivo when such prodrug is administered to a mammalian subject. Since prodrugs are known to enhance numerous desirable qualities of pharmaceuticals (e.g., solubility, bioavailability, manufacturing, etc.) the compounds of the present invention may be delivered in prodrug form. Thus, the present invention contemplates prodrugs of the compounds of the present invention, compositions containing the same and methods of treating diseases and disorders with such prodrugs. Prodrugs of a compound of the present invention, for example Formula I, may be prepared by modifying functional groups present in the compound in such a way that the modifications are cleaved, either in routine manipulation or in vivo, to the parent compound. Accordingly, prodrugs include, for example, compounds of the present invention wherein a hydroxy, amino, or carboxy group is bonded to any group that, when the prodrug is administered to a mammalian subject, cleaves to form a free hydroxyl, free amino, or carboxylic acid, respectively. Examples include, but are not limited to, the residue of an amino acid after the hydroxyl group of the carboxyl group is removed acetate, formate and benzoate derivatives of alcohol and amine functional groups; and alkyl, carbocyclic, aryl and alkylaryl esters such as methyl, ethyl, propyl, iso-propyl, butyl, isobutyl, sec-butyl, tert-butyl, cyclopropyl, phenyl, benzyl and phenethyl esters and the like.

Certain abbreviations used to delineate the results below are defined as follows: “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar and “nm” denotes nanometer.

“Microparticle suspension” is defined as a suspension in an aqueous solution of emulsified particles containing pharmaceutical agents. A minimum of about ½ of the particles has a particle size less than 200 microns.

“Biodegradable polymer matrix” is defined as a matrix of polymer materials that break-down or degrade when the material is placed in-vivo and where the degradation effects at least in part the rate of release of the pharmaceutical agents.

Active Ingredients

One embodiment of the present invention is the fused pyrrolocarbazoles represented by Formula I:

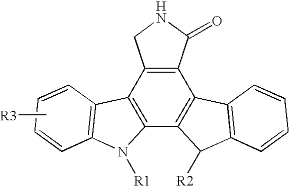

wherein:

R1 and R2 are the same or different and are independently selected from —H, or alkyl of 1-8 carbons, preferably an alkyl of 1-4 carbons, substituted with —OH, or —OR4 where R4 is an alkyl of 1-4 carbons, aryl, preferably phenyl or naphthyl, or the residue of an amino acid after the hydroxyl group of the carboxyl group is removed; and

R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6. R5 is alkyl of 1-4 carbons or aryl, preferably phenyl or naphthyl. R6 is H, alkyl of 1-4 carbons, aryl of 6-10 carbons, preferably phenyl or naphthyl, or heteroaryl. R7 is H or alkyl of 1-4 carbons. R8 is the residue of an amino acid after the hydroxyl group of the carboxyl group is removed; n is an integer of 1-4; and y is 1 or 2.

In certain preferred embodiments, the compounds of Formula I are those of Formula II:

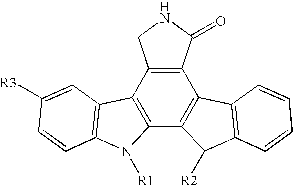

wherein R1, R2 and R3 are as defined for Formula I above.

In certain referred embodiments, R1 is an alkyl of 1-4 carbons, substituted with —OH or —OR4 where R4 is an alkyl of 1-4 carbons (inclusive), aryl, preferably phenyl or naphthyl, or the residue of an amino acid after the hydroxyl group of the carboxyl group is removed. R₂ is H; and R₃ is —CH₂OH; —CH₂OR7; —(CH₂)ₙSR5; —(CH₂)ₙS(O)_{y}R5; —CH₂SR₅; or alkyl of 1-8 carbons, preferably an alkyl of 1-4 carbons, substituted with —OH, —OR5, —OR8, —CH₂OR7, —S(O)_{y}R6 or —SR6. R5, R6, R7 and R8 are as defined for Formula I above.

In certain other preferred embodiments, R1 is —CH₂CH₂CH₂OH or —CH₂CH₂CH₂—OCOCH₂N(CH₃)₂, R2 is H and R3 is —CH₂OR₇; wherein R7 is alkyl of 1-4 carbons.

In certain even further preferred embodiments the fused pyrrolocarbazoles of Formula I and/or Formula II are those represented in Table I.

Particularly preferred compounds of Table I include compounds 1, 2, 3, 4, 5, 6 and 21 with compounds 2 and 21 being most preferred.

Pharmaceutically acceptable salts of the fused pyrrolocarbazoles of the present invention also fall within the scope of the compounds as disclosed herein. Some examples of acid addition salts include the hydrochloride, sulfate and phosphate salts of a base containing organic molecule. Some examples of organic acid addition salt such as acetate, maleate, fumarate, tartrate and citrate salts of the base containing organic molecule. Examples of pharmaceutically acceptable metal salts are alkali metal salts such as sodium salt and potassium salt, alkaline earth metal salts such as magnesium salt and calcium salt, aluminum salt and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine and phenylalanine.

Therapeutic and Prophylactic Indications

In one embodiment, there is a pharmaceutical delivery system for treating inflammation-mediated condition of the eye, for example edema. The pharmaceutical delivery system comprises, a fused pyrrolocarbazole, an indenocarbazole, an indolocarbazole, a compound of Formula I, a compound of Formula II or a compound of Table I and a biodegradable polymer matrix according to any one of the embodiments disclosed herein.

In one embodiment, there is a pharmaceutical delivery system for inhibiting VEGFR kinase activity in the eye. The pharmaceutical delivery system comprises, a fused pyrrolocarbazole, an indenocarbazole, an indolocarbazole, a compound of Formula I, a compound of Formula II or a compound of Table I and a biodegradable polymer matrix according to any one of the embodiments disclosed herein.

In one embodiment, there is a pharmaceutical delivery system for treating angiogenesis disorders in the eye of a patient. The pharmaceutical delivery system comprises, a fused pyrrolocarbazole, an indenocarbazole, an indolocarbazole, a compound of Formula I, a compound of Formula II or a compound of Table I and a polymer matrix material according to any one of the embodiments disclosed herein.

In one embodiment, there is a method of treating inflammation-mediated condition of the eye (for example edema). The method comprising administering to the eye of a patient a pharmaceutical delivery system comprises, a fused pyrrolocarbazole, an indenocarbazole, an indolocarbazole, a compound of Formula I, a compound of Formula II or a compound of Table I and a polymer matrix material according to any one of the embodiments disclosed herein.

In one embodiment, there is a method for inhibiting VEGFR kinase activity in the eye of a patient. The method comprises administering to the eye of a patient a pharmaceutical delivery system comprising, a fused pyrrolocarbazole, an indenocarbazole, an indolocarbazole, a compound of Formula I, a compound of Formula II or a compound of Table I and a biodegradable polymer matrix according to any one of the embodiments disclosed herein.

In one embodiment, there is a pharmaceutical delivery system for treating angiogenesis disorders in the eye of a patient. The method comprises administering to a patient a pharmaceutical delivery system comprises, a fused pyrrolocarbazole, an indenocarbazole, an indolocarbazole, a compound of Formula I, a compound of Formula II or a compound of Table I and a biodegradable polymer matrix according to any one of the embodiments disclosed herein.

In one embodiment, there is a pharmaceutical delivery system for treating retinopathy, diabetic retinopathy or macular degeneration in the eye of a patient. The pharmaceutical delivery system comprises, a fused pyrrolocarbazole, an indenocarbazole, an indolocarbazole, a compound of Formula I, a compound of Formula II or a compound of Table I and a biodegradable polymer matrix according to any one of the embodiments disclosed herein.

In one embodiment, there is a pharmaceutical delivery system for treating retinopathy, diabetic retinopathy or macular degeneration in the eye of a patient. The pharmaceutical delivery system comprises, a fused pyrrolocarbazole, an indenocarbazole, an indolocarbazole, a compound of Formula I, a compound of Formula II or a compound of Table I having a single or multiple crystalline morphology and a biodegradable polymer matrix according to any one of the embodiments disclosed herein.

The fused pyrrolocarbazoles of the present invention have important functional pharmacological activities, which find utility in a variety of settings, including both research and therapeutic arenas. For ease of presentation and in order not to limit the range of utilities for, which these compounds can be characterized, we generally describe the activities of the fused pyrrolocarbazoles in ocular tissue including inhibition of enzymatic activity such as the enzymatic kinase activity of VEGFR1 and VEGFR2; inhibition of disorders due to angiogenesis or neovascularization; and inhibition of inflammation-associated responses.

Synthesis of Fused Pyrrolocarbazoles

The present invention also provides a method for preparing the fused pyrrolocarbazoles of the present invention. The compounds of the present invention may be prepared in a number of ways well known to those skilled in the art. Specifically, Compounds A and B were prepared according to the disclosure of U.S. Pat. Nos. 5,475,110, 5,591,855, 5,594,009, 5,616, 724, 5,705,511 and 6,630,500, which is incorporated herein by reference in its entirety.

It will be appreciated that the compounds of the present invention may contain one or more asymmetrically substituted carbon atoms and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare such optically active forms. For example, mixtures of stereoisomers may be separated by standard techniques including, but not limited to, resolution of racemic forms, normal, reverse-phase and chiral chromatography, preferential salt formation, recrystallization and the like, or by chiral synthesis either from active starting materials or by deliberate chiral synthesis of target centers.

As will be readily understood, functional groups present on the compounds of the present invention may contain protecting groups. For example, the amino acid side chain substituents of the compounds can be substituted with protecting groups such as benzyloxycarbonyl or tert-butoxycarbonyl groups. Protecting groups are known per se as chemical functional groups that can be selectively appended to and removed from functionalities, such as hydroxyl groups and carboxyl groups. These groups are present in a chemical compound to render such functionality inert to chemical reaction conditions to, which the compound is exposed. Any of a variety of protecting groups may be employed with the present invention. Preferred protecting groups include the benzyloxycarbonyl (Cbz; Z) group and the tert-butyloxycarbonyl (Boc) group. Other preferred protecting groups according to the invention may be found in Greene, T. W. and Wuts, P. G. M., “Protective Groups in Organic Synthesis” 2d. Ed., Wiley & Sons, 1991.

Combination Therapies

The fused pyrrolocarbazole may be administered in combination with one or more additional pharmaceutical agents, such as the individual compounds and therapeutic agent within one or more of the therapeutic classes selected from the group comprising anti-metabolites, anti-biotics, antibacterials, antifungal antibiotics, synthetic antifungals, steroids, anti-proliferative agents, matrix metalloproteinase inhibitors, thrombolytic agents, anti-neoplastic agents, non-steriodal anti-inflammatories (NSAIDS) and retinoids.

Additionally, the fused pyrrolocarbazoles of the present invention optionally can be used in combination with one or more anti-angiogenesis agents including but not limited to other tyrosine kinase inhibitors, inhibitors of growth factors, inhibitors of Tie-2, inhibitors of angiopoetin.

Additionally, the fused pyrrolocarbazoles of the present invention can be used in combination with agents that promote survival of retinal cells including, but not limited to, neurons, glia and retinal pigment epithelium, such as neurotrophic factors, anti-apoptosis agents, and anti-caspase agents.

The fused pyrrolocarbazole or the fused pyrrolocarbazole and the additional pharmaceutical agent(s) are preferably from about 10 to 90% by weight of the pharmaceutical delivery system. More preferably, the fused pyrrolocarbazole or the fused pyrrolocarbazole and the additional pharmaceutical agent(s) are from about 50 to about 80% by weight of the pharmaceutical delivery system. In a preferred embodiment, the agent comprises about 50% by weight of the pharmaceutical delivery system. In a particularly preferred embodiment, the agent comprises about 70% by weight of the pharmaceutical delivery system.

Biodegradable Polymer Matrices

In another embodiment, there is a pharmaceutical delivery system that is made of a biodegradable polymers matrix comprising a biodegradable polymer and a fused pyrrolocarbazole. In one embodiment, the biodegradable polymer matrix is a microparticle (i.e. microsphere) suspension and includes particles such as nanoparticles (i.e. nanospheres).

In still another embodiment, the amount of fused pyrrolocarbazole is present in the biodegradable polymer matrix is a minimum of about 10 wt. % and a maximum of about 90 wt. % based upon the total weight of the biodegradable polymer matrix. Typically, the amount of fused pyrrolocarbazole is present in the biodegradable polymer matrix is a minimum of about 20 wt. %, about 30 wt. %, about 40 wt. % or about 50 wt. % and/or a maximum of about 85 wt. %, 80 wt. %, 75 wt. %, about 70 wt. %, about 65 wt. %, about 60 wt. %, about 55 wt. % or about 50 wt. % based upon the total weight of the biodegradable polymer matrix. Generally, the biodegradable polymer is selected from the group consisting of polylactic acid, polylactate, polyglycolic acid, polyglycolate and copolymers thereof.

Optionally, the biodegradable polymer is a poly(lactic-co-glycolic acid) polymer system or poly(lactate-co-glycolate) polymer system wherein the ratio of lactic and/or lactate monomers to glycolic and/or glycolate monomers is any possible value having a minimum of 0:100 and/or a maximum of 100:0. Typically, the ratio of lactic and/or lactate monomers to glycolic and/or glycolate monomers is a minimum of about 0:100, about 30:70, about 35:65, about 40:60, about 45:65 or about 50:50 and/or a maximum of about 100:0, about 70:30, about 65:35, about 60:30, about 55:45 or about 50:50.

The biodegradable polymer matrices of the invention are formulated with particles of the fused pyrrolocarbazole mixed within the bioerodible polymer matrix. Release of the agent is achieved by erosion of the polymer followed by exposure of previously entrapped agent particles to the vitreous and subsequent dissolution and release of agent. The release kinetics achieved by this form of pharmaceutical agent release are different than that achieved through formulations, which release pharmaceutical agent through polymer swelling, such as with hydrogels such as methylcellulose. In that case, the pharmaceutical agent is not released through polymer erosion, but through polymer swelling, which releases pharmaceutical agent as liquid diffuses through the pathways exposed. The parameters, which determine the release kinetics include the size of the pharmaceutical agent particles, the water solubility of the pharmaceutical agent, crystal structure or polymorphic composition, the ratio of pharmaceutical agent to polymer, the method of manufacture, the surface area exposed and the erosion rate of the polymer.

In one embodiment, the pharmaceutical delivery system is multilayered with differing drug concentrations. In one embodiment, the pharmaceutical delivery system has an inner core that has a first concentration of drug(s) and a second outer layer that has a higher concentration of drug(s) enabling, in some instances, a relatively high initial dose followed by a relatively lower dose. In another embodiment, the first concentration is higher than the second concentration thus enabling, in some instances, a relatively low initial dose followed by a relatively higher dose.

In another embodiment, the weight average molecular weight of the biodegradable polymer is a minimum of about 1 kD and a maximum of about 1000 kD. Typically, the weight average molecular weight of the biodegradable polymer is a minimum of about 3 kD, about 5 kD, about 7 kD, about 10 kD or about 15 kD and or a maximum of about 100 kD, about 50 kD, about 20 kD, about 15 kD, about 10 kD. In one embodiment, the weight average molecular weight of the biodegradable polymer is about 7 kD.

The biodegradable polymer matrices are preferably monolithic, i.e. having the fused pyrrolocarbazole homogenously distributed through the polymeric matrix. The selection of the polymeric composition to be employed will vary with the desired release kinetics, patient tolerance, the nature of the disease to be treated and the like. Characteristics of the polymers will include biodegradability at the site of implantation, compatibility with the agent of interest, ease of encapsulation, water insolubility and the like. Preferably, the polymeric matrix will not be fully degraded until the pharmaceutical agent load has been released.

Biodegradable polymeric compositions, which may be employed may be organic esters or ethers, which when degraded result in physiologically acceptable degradation products, including the monomers. Anhydrides, amides, orthoesters or the like, by themselves or in combination with other monomers, may find use. The polymers of one embodiment are condensation polymers. Alternatively, the polymers are cross-linked or non-cross-linked, usually not more than lightly cross-linked, generally less than about 5%, usually less than about 1% after polymerization. Oxygen may be present as oxy, e.g., hydroxy or ether, carbonyl, e.g., non-oxo-carbonyl, such as carboxylic acid ester and the like. Nitrogen may be present as amide, cyano and amino. The biodegrable polymers set forth in Heller, “Biodegrable Polymers in Controlled Drug Delivery,” CRC Critical Reviews in Therapeutic Drug Carrier Systems, vol. 1, CRC Press, Boca Raton, Fla. (1987), are used in one embodiment. In one embodiment, the polymers are stable to sterilization. Thus, irradiation does not cause decomposition to the extent that the physical properties of the polymers change in a substantial way. By change in a substantial way as used in this paragraph, it is meant that the physical properties of the polymers do not change to the extent that they cannot function for its intended purpose.

Of particular interest are polymers of hydroxyaliphatic carboxylic acids, either homo- or copolymers and polysaccharides. Included among the polyesters of interest are polymers of D-lactic acid, L-lactic acid, racemic lactic acid, glycolic acid, polycaprolactone and combinations thereof. By employing the L-lactate or D-lactate, a slowly biodegrading polymer is achieved, while degradation is substantially enhanced with the racemate.

The size of the bulk polymer particles is preferably a minimum of about 1 microns, about 2 microns, about 5 microns, about 9 microns, or about 10 microns in diameter and/or a maximum of about 100 microns, about 50 microns, about 20 microns, about 12 microns or about 10 microns in diameter.

Likewise, the size of the drug particles is preferably a minimum of about 1 microns, about 2 microns, about 5 microns, about 9 microns, or about 10 microns in diameter and/or a maximum of about 100 microns, about 50 microns, about 20 microns, about 12 microns or about 10 microns in diameter. In one embodiment, the biodegradable polymer matrix comprises a minimum of about 5 wt. %, about 10 wt. %, about 15 wt. % or about 20 wt. % polymer matrix material. The biodegradable polymer matrix comprises a maximum of about 40 wt. %, about 30 wt. %, about 25 wt. % or about 20 wt. % polymer matrix material.

In an embodiment, the biodegradable polymer matrix comprises a minimum of about 40 wt. %, about 50 wt. %, about 60 wt. % or about 75 wt. % fused pyrrolocarbazole and in particular compounds 2 and/or 21. The biodegradable polymer matrix comprises a maximum of about 90 wt. %, about 85 wt. %, about 80 wt. % or about 75 wt. % fused pyrrolocarbazole and in particular compounds 2 and/or 21.

In another embodiment, the biodegradable polymer matrix comprises a minimum of about 1 wt %, about 2 wt. %, about 4 wt. % and preferably about 5 wt. % of a vitamin derived surfactant and/or a maximum of about 10 wt. %, about 8 wt. %, about 7 wt. % and preferably about 5 wt. % a vitamin derived surfactant.

Two very useful vitamin derived surfactants are D-alpha-tocopheryl polyethylene glycol 1000 succinate (Vitamin E TPGS), and its amide analogue (Vitamin E TPGSA). Vitamin E TPGS is a polyethylene glycol (PEG) ester of D-alpha-tocopheryl acid succinate, where the polyethylene glycol (PEG) molecular weight is about 1000. D-alpha-tocopheryl acid succinate is, in turn, a succinic acid ester of D-alpha-tocopherol, Vitamin E. Vitamin E TPGSA is a polyethylene glycol (PEG) amide of D-alpha-tocopheryl acid succinate, containing an amide bond between the PEG chain and the distal succinic acid free acid group, and where the PEG molecular weight is about 1000.

Among the biodegradable polymer matricies of interest there is a polymer matrix made from polysaccharide. Polysaccharides of interest are calcium alginate and functionalized celluloses, particularly carboxymethylcellulose esters characterized by being biodegradable, water insoluble, a molecular weight of about 5 kD to about 500 kD.

Generally, additional release modulators such as those described in U.S. Pat. No. 5,869,079 are included in the biodegradable polymer matrices. The amount of release modulator employed will be dependent on the desired release profile, the activity of the modulator and the release profile of the fused pyrrolocarbazole in the absence of modulator.

Other agents may be employed in the formulation for a variety of purposes. For example, buffering agents and preservatives may be employed. Water-soluble preservatives, which may be employed include sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. These agents may be present in individual amounts of from about 0.001 wt. % to about 5 wt. % and preferably about 0.01 wt. % to about 2 wt. %. Suitable water-soluble buffering agents that may be employed are sodium carbonate, sodium borate, sodium phosphate, sodium acetate, sodium bicarbonate, etc., as approved by the FDA for the desired route of administration. These agents may be present in amounts sufficient to maintain a pH of the system of between 2 to 9 and preferably 4 to 8. As such, the buffering agent may be as much as 5 wt. % of the total composition. Electrolytes such as sodium chloride and potassium chloride may also be included in the formulation. Where the buffering agent or enhancer is hydrophilic, it may also act as a release accelerator. Hydrophilic additives act to increase the release rates through faster dissolution of the material surrounding the pharmaceutical agent particles, which increases the surface area of the pharmaceutical agent exposed, thereby increasing the rate of bioerosion. Similarly, a hydrophobic buffering agent or enhancer dissolve more slowly, slowing the exposure of pharmaceutical agent particles and thereby slowing the rate of bioerosion.

The proportions of fused pyrrolocarbazole, polymer and any other modifiers are determined, typically, by formulating several biodegradable polymer matrices with varying proportions. A USP approved method for dissolution or release test can be used to measure the rate of release (USP 23; NF 18 (1995) pp. 1790-1798). For example, using the infinite sink method, a weighed sample of the pharmaceutical delivery system is added to a measured volume of a solution containing 0.9% NaCl in water, where the solution volume will be such that the pharmaceutical agent concentration is less than 5% of saturation after release. The mixture is maintained at 37° C. and stirred slowly to maintain the biodegradable polymer matrices in suspension. The appearance of the dissolved pharmaceutical agent as a function of time, generally, is followed by various methods known in the art, such as spectrophotometrically, HPLC, mass spectroscopy, etc. until the absorbance becomes constant or until greater than 90% of the pharmaceutical agent has been released.

The release kinetics of the biodegradable pharmaceutical delivery systems of the invention are dependent in part on the surface area of the devices. Larger surface area exposes more polymer to the surrounding microenvironment, for example, the vitreous, causing faster erosion and dissolution of the particles of pharmaceutical agent entrapped by the polymer. The size and form of the biodegradable polymer matrix can be used to control the rate of release, period of treatment and pharmaceutical agent concentration at the site of implantation. Larger biodegradable polymer matrices will deliver a proportionately larger dose, but depending on the surface to mass ratio, may have a slower release rate. The biodegradable polymer matrices may be particles, sheets, patches, plaques, films, discs, fibers, microcapsules and the like and may be of any size or shape compatible with the selected site of insertion, as long as the biodegradable polymer matrices have the desired release kinetics. Preferably, the biodegradable polymer matrix to be inserted is formulated as a single particle. Preferably, the biodegradable polymer matrix will not migrate from the insertion site following implantation. The upper limit for the size of the biodegradable polymer matrix will be determined by factors such as the desired release kinetics, toleration for the biodegradable polymer matrix, size limitations on insertion, ease of handling, etc. The vitreous chamber is able to accommodate relatively large biodegradable polymer matrices of varying geometries, having diameters of 1 to 3 mm. In a preferred embodiment, the biodegradable polymer matrix is a cylindrical pellet (e.g., rod) that has a maximum length of about 10 mm, about 5 mm or about 2 mm. The diameter of the device is preferably a maximum of about 2 mm, about 1 mm, about 0.75 mm or about 0.4 mm. Preferably, the device is about 2 mm by 0.4 mm.

The biodegradable polymer matrices will also preferably be at least somewhat flexible so as to facilitate both insertion of the biodegradable polymer matrix in the vitreous and accommodation of the biodegradable polymer matrix. The total weight of the biodegradable polymer matrix delivery system is preferably a minimum of about 100 μg, about 250 μg, about 500 μg or about 1000 μg. In one embodiment, the biodegradable polymer matrix delivery system has a maximum weight of about 5000 μg, about 2000 μg, about 1000 μg, about 750 μg or about 500 μg.

Methods for Making the Biodegradable Polymer Matrices

Various techniques may be employed to produce the biodegradable polymer matrices. Useful techniques include phase separation methods, interfacial methods, extrusion methods, compression methods, molding methods, injection molding methods, heat press methods and the like.

Choice of the technique and manipulation of the technique parameters employed to produce the biodegradable polymer matrices can influence the release rates of the pharmaceutical agent. Room temperature compression methods result in an biodegradable polymer matrix with discrete microparticles of pharmaceutical agent and polymer interspersed. Extrusion methods result in biodegradable polymer matrices with a progressively more homogenous dispersion of the pharmaceutical agent within the polymer, as the production temperature is increased. When using extrusion methods, the polymer and pharmaceutical agent are chosen to as to be stable at the temperatures required for manufacturing, usually at least about 85° C. Extrusion methods use temperatures of about 25° C. to about 150° C., more preferably about 65° C. to about 130° C. Generally, compression methods yield biodegradable polymer matrices with faster release rates than extrusion methods and higher temperatures yield biodegradable polymer matrices with slower release rates.

In a preferred embodiment, compression methods are used to produce the biodegradable polymer matrices of the invention. Preferably, compression methods use pressure that are a minimum of about 50 psi, about 70 psi or about 75 psi and/or a maximum of about 150 psi, about 100 psi or about 80 psi. The compression method typically has a temperature that is a minimum of about 0° C. or about 25° C. and/or a maximum of about 115° C. or about 75° C. or about 50° C. Most preferably, the temperature is about ambient conditions (i.e., 25° C.). Biodegradable polymer matrices that are produced by extrusion methods are heated to a temperature that is a minimum of about 60° C. or about 130° C. and/or a maximum of about 150° C. or about 130° C. for pharmaceutical agent/polymer mixing. The extrusion method mixes at this temperature for a time period that is a minimum of about 0 to 1 hour, 0 to 30 minutes, 5-15 minutes, preferably about 10 minutes, preferably about 0 to 5 min. Preferably, the biodegradable polymer matrices are then extruded at a temperature from about 60° C. to about 130° C., more preferably about 75° C.

Kits for the Administration of the Pharmaceutical Delivery System

In another aspect of the invention, kits for treating angiogenesis-mediated conditions of the eye are provided, comprising: (a) one or more pharmaceutical delivery systems disclosed herein and (b) instructions for use.

In another aspect of the invention, kits for treating one or more inflammatory conditions of the eye (for example edema) that are identified herein that are provided, comprising: (a) one or more pharmaceutical delivery systems disclosed herein and (b) instructions for use.

Method of Administering the Pharmaceutical Delivery System

The pharmaceutical delivery system is typically inserted into the eye by a trocar following making an incision in the sclera sized to receive the trocar. The pharmaceutical delivery system may also be administered into the eye by injection via a needle. The method of placement may influence the pharmaceutical agent release kinetics. For example, implanting the device with a trocar may result in placement of the device deeper within the vitreous than placement by forceps, which may result in the biodegradable polymer matrix being closer to the edge of the vitreous. The location of the implanted device may influence the concentration gradients of pharmaceutical agent surrounding the device and thus influence the release rates (e.g., a device placed closer to the edge of the vitreous will result in a slower release rate). One example of a placement device is found in U.S. Patent Publ. No. 2003/0135153, which is incorporated herein by reference in its entirety.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A pharmaceutical delivery system comprising a fused pyrrolocarbazole and a biodegradable polymer matrix configured to be inserted into the eye of the patient.

2. The pharmaceutical delivery system of claim 1, wherein the fused pyrrolocarbazole is selected from the group consisting of an indolocarbazole and an indenocarbazole and mixtures thereof.

3. The pharmaceutical delivery system of claim 1, wherein the fused pyrrolocarbazole is a compound defined by the following formula and salts thereof and prodrugs thereof and mixtures of the compound, salt and prodrug thereof: 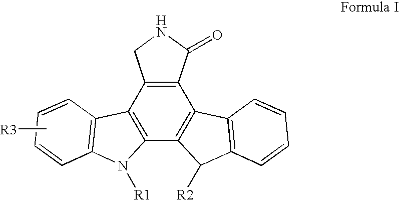
wherein:
R1 and R2 are the same or different and are independently selected from —H, or alkyl of 1-8 carbons, preferably an alkyl of 1-4 carbons, substituted with —OH, or —OR4 where R4 is an alkyl of 1-4 carbons, aryl, preferably phenyl or naphthyl, or the residue of an amino acid after the hydroxyl group of the carboxyl group is removed; and
R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6; and wherein
R5 is alkyl of 1-4 carbons or aryl, preferably phenyl or naphthyl;
R6 is H, alkyl of 1-4 carbons, aryl of 6-10 carbons, preferably phenyl or naphthyl, or heteroaryl;
R7 is H or alkyl of 1-4 carbons;
R8 is the residue of an amino acid after the hydroxyl group of the carboxyl group is removed;
n is an integer of 1-4; and
y is 1 or 2, with the proviso that when R1 is —(CH₂)₃OH and R2 is H, then R3 cannot be —CH₂OH,
—CH₂OCH₂CH₃, or —CH₂SCH₂CH₃.

4. The pharmaceutical delivery system of claim 1, wherein the fused pyrrolocarbazole is one or more compounds defined by Formula II and salts thereof and prodrugs thereof and mixtures of the compounds, salts and prodrugs thereof: 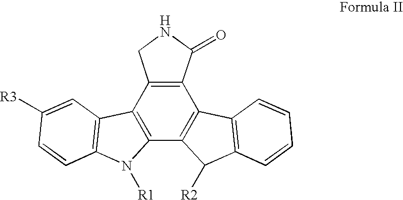
R₁ and R₂ are the same or different and are independently selected from H, or alkyl of 1-8 carbons, substituted with —H, —OH or —OR4 where R4 is an alkyl of 1-4 carbons, aryl or the residue of an amino acid after the hydroxyl group of the carboxyl group is removed;
and R3 is —CH₂OH; —CH₂OR7; —(CH₂)nSR5; —(CH₂)nSOyR5; —CH₂SR₅; or alkyl of 1-8 carbons substituted with —OH, —OR5, —OR8, —CH₂OR7, —S(O)yR6 or —SR6; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and y is 1 or 2.

5. The pharmaceutical delivery system of claim 4, wherein R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)yR6 or —SR8; and wherein R5 is alkyl of 1-4 carbons or aryl; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

6. The pharmaceutical delivery system of claim 4, wherein R1 is —CH₂CH₂CH₂OH or —CH₂CH₂CH₂OCOCH₂N(CH₃)₂; R2 is H; and R3 is —CH₂OR7 wherein R7 is alkyl of 1-4 carbons.

7. The pharmaceutical delivery system of claim 1, wherein the fused pyrrolocarbazole is selected from the groups consisting of compounds represented in Table I and salts thereof and prodrugs thereof and mixtures of the salts and prodrugs thereof:
TABLE

| CMPD | | | |
|---|---|---|---|
| NO | R1 | R2 | R3 |
| 1 | —CH₂CH₂CH₂OH | —H | CH₂OCH₃ |
| 2 | —CH₂CH₂CH₂OH | —H | —CH₂OCH(CH₃)₂ |
| 3 | —CH₂CH₂CH₂OH | —H | —CH₂O—CH(CH₃)CH₂CH₃ |
| 4 | —CH₂CH₂CH₂OH | —H | (S)—CH₂O—CH(CH₃)CH₂CH₃ |
| 5 | —CH₂CH₂CH₂OH | —H | (R)—CH₂O—CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂CH₂CH₃ |
| 9 | —CH₂CH₂CH₂OH | —H | —CH(CH₃)OCH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —H | | —CH₂OCH₂CH₃ |
| 14 | —CH₂CH₂CH₂OH | —H | —CH(CH₃)O—CH₂CH₂CH₂CH₃ |
| 15 | —CH₂CH₂CH₂OH | —H | —CH(CH₃)O—CH(CH₃)₂ |
| 16 | —CH₂CH₂CH₂OH | —H | —CH₂OC(CH₃)₃ |
| 17 | —CH₂CH₂CH₂OCO—CH₂NH₂ | —H | —CH₂OCH(CH₃)₂ |
| 18 | —CH₂CH₂CH₂OCO—CH₂NH₂—CH₂CH₂CH₂CH₂NH₂ | —H | —CH₂OCH(CH₃)₂ |
| 19 | —CH₂CH₂CH₂OCOCH₂—CH₂NH₂ | —H | —CH₂OCH(CH₃)₂ |
| 20 | —CH₂CH₂CH₂OCOCH₂—CH₂CH₂N(CH₃)₂ | —H | —CH₂OCH(CH₃)₂ |
| 21 | —CH₂CH₂CH₂OCO—CH₂N(CH₂)₂ | —H | —CH₂OCH(CH₃)₂ |
| 22 | —CH₂CH₂CH₂OCO—CH₂CH₂CH₂ | —H | —CH₂OCH(CH₃)₂ |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−)—CH(OCH₃)CH₃ |
| 34 | —CH₂CH₂CH₂OH | —CH₂OH | —CH₂OCH(CH₃)₂ |

8. The pharmaceutical delivery system of claim 1, wherein the fused pyrrolocarbazole is of the following formula and salts thereof and prodrugs thereof and mixtures of the compound, salts and prodrugs therof:

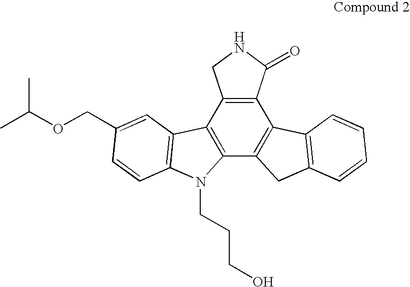

9. The pharmaceutical delivery system of claim 1, wherein the fused pyrrolocarbazole is a compound of the following formula and salts thereof and prodrugs thereof and mixtures of the compound, salts and/or prodrugs thereof:

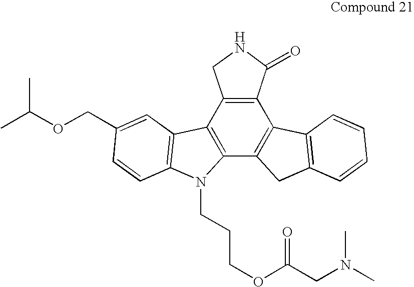

10. The pharmaceutical delivery system of claim 1, wherein the biodegradable polymer matrix is in the form of an implant.

11. The pharmaceutical delivery system of claim 1, wherein the biodegradable polymer is in the form of microspheres.

12. The pharmaceutical delivery system of claim 1, wherein the amount of active ingredient is present in the biodegradable polymer matrix is a minimum of about 10 wt. % and a maximum of about 80 wt. % based upon the total weight of the biodegradable polymer matrix.

13. The pharmaceutical delivery system of claim 1, wherein the biodegradable polymer is selected from the group consisting of polylactic acid, polylactate polyglycolic acid, polyglycolate and copolymers thereof.

14. The pharmaceutical delivery system of claim 1, wherein the biodegradable polymer is a poly(lactic-co-glycolic acid) polymer system or poly(lactate-co-glycolate) polymer system wherein the ratio of lactic and/or lactate monomers to glycolic and/or glycolate monomers is a minimum of about 30:70 and a maximum of about 70:30.

15. The pharmaceutical delivery system of claim 1, wherein the weight average molecular weight of the biodegradable polymer is a minimum of about 1 kD and a maximum of about 1000 kD.

16. The pharmaceutical delivery system of claim 1, wherein the system is configured to maintain the concentration of fused pyrrolocarbazole in the vitreous that is a minimum of about 10 ng/ml.

17. The pharmaceutical delivery system of claim 1, that is configured to maintain the effective concentration of fused pyrrolocarbazole in the vitreous that is at least about 50 times greater than the concentration of the fused pyrrolocarbazole in the blood of the patient.

18. The pharmaceutical delivery system of claim 1, wherein the effective concentration of fused pyrrolocarbazole in the vitreous of is maintained for a minimum of 6 weeks.

19. The pharmaceutical delivery system of claim 1, wherein the anti-angiogenesis agent is released from the pharmaceutical delivery system at rate that is a minimum of about 5 ng per day and a maximum of about 1 mg per day.

20. A method for treating angiogenic disorders in the eye of a patient, which comprises administering to a host in need of such treatment a pharmaceutical delivery system comprising a biodegradable polymer matrix and a therapeutically effective amount of a fused pyrrolocarbazole.

21. The method of claim 20, wherein the fused pyrrolocarbazole is selected from the group consisting of an indolocarbazole and an indenocarbazole and mixtures thereof.

22. The method of claim 20, wherein the fused pyrrolocarbazole is defined by the following Formula I and salts thereof and prodrugs thereof: 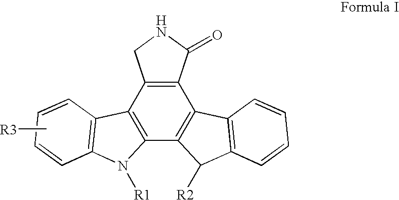
R1 and R2 are the same or different and are independently selected from H, or alkyl of 1-8 carbons, substituted with —OH, or —OR4 where R4 is an alkyl of 1-4 carbons, aryl or the residue of an amino acid after the hydroxyl group of the carboxyl group is removed; and R3 is —CH₂OH; —CH₂OR7; —(CH₂)nSR5; —(CH₂)nS(O)yR5; —CH₂SR5; or alkyl of 1-8 carbons substituted with —OH, —OR5, —OR8, —CH₂OR7, —S(O)yR6 or —SR8; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and y is 1 or 2; with the proviso that when R1 is (CH₂)₃OH and R2 is H, then R3 cannot be —CH₂OH, alkyl of 1-8 carbons substituted with —OH or —SR8, wherein R6 is alkyl of 1-4 carbons; —(CH₂)nSR5, wherein n is 1 and R5 is alkyl of 1-4 carbons; or —CH₂SR5, wherein R5 is alkyl of 1-4 carbons.

23. The method of claim 20, wherein the fused pyrrolocarbazole is defined by the following Formula II and salts thereof and prodrugs thereof: 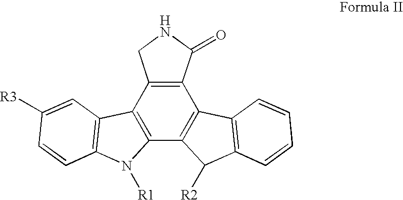
R₁ and R₂ are the same or different and are independently selected from H, or alkyl of 1-8 carbons, substituted with —H, —OH or —OR4 where R4 is an alkyl of 1-4 carbons, aryl or the residue of an amino acid after the hydroxyl group of the carboxyl group is removed; and R3 is —CH₂OH; —CH₂OR7; —(CH₂)nSR5; —(CH₂)nSOyR5; —CH₂SR₅; or alkyl of 1-8 carbons substituted with —OH, —OR5, —OR8, —CH₂OR7, —S(O)yR6 or —SR6; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and y is 1 or 2; with the proviso that when R1 is —(CH₂)₃OH and R2 is —H, then R3 cannot be —CH₂OH, —CH₂OCH₂CH₃, or —CH₂SCH₂CH₃.

24. The method of claim 23, wherein R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)yR6 or —SR8; and wherein R5 is alkyl of 1-4 carbons or aryl; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

25. The method of claim 23, wherein R1 is —CH₂CH₂CH₂OH or —CH₂CH₂CH₂OCOCH₂N(CH₃)₂; R2 is H; and R3 is —CH₂OR7 wherein R7 is alkyl of 1-4 carbons.

26. The method of claim 20 wherein the fused pyrrolocarbazole is selected from the group consisting of the compounds represented in Table I and salts thereof and prodrugs thereof and mixtures of such compounds, salts and/or prodrugs thereof:

| CMPD | | | |
|---|---|---|---|
| NO | R1 | R2 | R3 |
| 1 | —CH₂CH₂CH₂OH | —H | —CH₂OCH₃ |
| 2 | —CH₂CH₂CH₂OH | —H | —CH₂OCH(CH₃)₂ |
| 3 | —CH₂CH₂CH₂OH | —H | —CH₂O—CH(CH₃)CH₂CH₃ |
| 4 | —CH₂CH₂CH₂OH | —H | (S)—CH₂O—CH(CH₃)CH₂CH₃ |
| 5 | —CH₂CH₂CH₂OH | —H | (R)—CH₂O—CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂CH₂CH₃ |
| 9 | —CH₂CH₂CH₂OH | —H | —CH(CH₃)OCH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —H | | —CH₂OCH₂CH₃ |
| 14 | —CH₂CH₂CH₂OH | —H | —CH(CH₃)O—CH₂CH₂CH₂CH₃ |
| 15 | —CH₂CH₂CH₂OH | —H | —CH(CH₃)O—CH(CH₃)₂ |
| 16 | —CH₂CH₂CH₂OH | —H | —CH₂OC(CH₃)₃ |
| 17 | —CH₂CH₂CH₂OCO—CH₂NH₂ | —H | —CH₂OCH(CH₃)₂ |
| 18 | —CH₂CH₂CH₂OCO—CH₂NH₂—CH₂CH₂CH₂CH₂NH₂ | —H | —CH₂OCH(CH₃)₂ |
| 19 | —CH₂CH₂CH₂OCOCH₂—CH₂NH₂ | —H | —CH₂OCH(CH₃)₂ |
| 20 | —CH₂CH₂CH₂OCOCH₂—CH₂CH₂N(CH₃)₂ | —H | —CH₂OCH(CH₃)₂ |
| 21 | —CH₂CH₂CH₂OCO—CH₂N(CH₂)₂ | —H | —CH₂OCH(CH₃)₂ |
| 22 | —CH₂CH₂CH₂OCO—CH₂CH₂CH₃ | —H | —CH₂OCH(CH₃)₂ |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−)—CH(OCH₃)CH₃ |
| 34 | —CH₂CH₂CH₂OH | —CH₂OH | —CH₂OCH(CH₃)₂ |

27. The method of claim 20, wherein the fused pyrrolocarbazole is a compound of the following formula and salts thereof and prodrugs thereof and mixtures of the compound, salts and/or prodrugs:

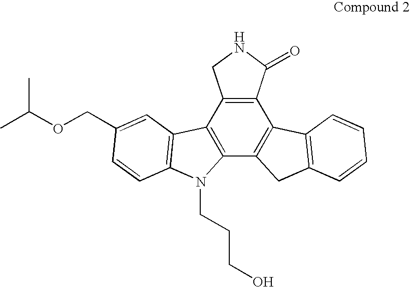

28. The method of claim 20, wherein the fused pyrrolocarbazole is of the following formula and salts and prodrugs thereof and mixtures of the compound, salts and/or prodrugs thereof:

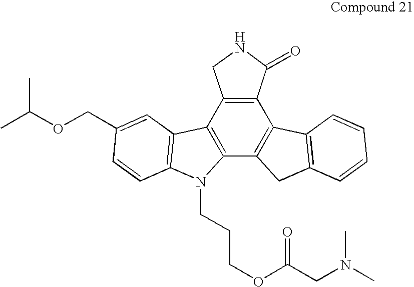

29. The method of claim 20, wherein the biodegradable polymer matrix is in the form of an implant.

30. The method of claim 20, wherein the biodegradable polymer matrix is in the form of a microsphere.

31. The method of claim 20 wherein the amount of active ingredient is present in the biodegradable polymer matrix is a minimum of about 10 wt. % and a maximum of about 80 wt. % based upon the total weight of the biodegradable polymer matrix.

32. The method of claim 20, wherein the biodegradable polymer is selected from the group consisting of polylactic acid, polylactate polyglycolic acid, polyglycolate and copolymers thereof.

33. The method of claim 20, wherein the biodegradable polymer is a poly(lactic-co-glycolic acid) polymer system or poly(lactate-co-glycolate) polymer system wherein the ratio of lactic and/or lactate monomers to glycolic and/or glycolate monomers is a minimum of about 30:70 and a maximum of about 70:30.

34. The method of claim 20, wherein the weight average molecular weight is a minimum of about 1 kD and a maximum of about 1000 kD.

35. The method of claim 20, wherein the pharmaceutical delivery system is configured to maintain the concentration of fused pyrrolocarbazole in the vitreous that is a minimum of about 10 ng/ml.

36. The method of claim 20, that is configured to maintain the effective concentration of fused pyrrolocarbazole in the vitreous that is at least about 50 times greater than the concentration of the fused pyrrolocarbazole in the blood of the patient.

37. The method of claim 20, wherein the effective concentration of fused pyrrolocarbazole in the vitreous of is maintained for a minimum of 6 weeks.

38. The method of claim 20, wherein the angiogenesis agent is released from the pharmaceutical delivery system at rate that is a minimum of about 5 ng per day and a maximum of about 1 mg per day.

39. The method of claim 19, wherein the angiogenesis agent is released from the pharmaceutical delivery system at rate that is a minimum of about 5 ng per day and a maximum of about 1 mg per day.

40. A method for treating an inflammatory disorder in the eye of a patient, which comprises administering to a host in need of such treatment a pharmaceutical delivery system comprising a drug-eluting polymer matrix and a therapeutically effective amount of a fused pyrrolocarbazole.

41. The method of claim 40, wherein the pharmaceutical delivery system is configured to maintain the concentration of fused pyrrolocarbazole in the vitreous that is a minimum of about 10 ng/ml.

42. The method of claim 40, that is configured to maintain the effective concentration of fused pyrrolocarbazole in the vitreous that is at least about 50 times greater than the concentration of the fused pyrrolocarbazole in the blood of the patient.

43. The method of claim 40, wherein the effective concentration of fused pyrrolocarbazole in the vitreous of is maintained for a minimum of 6 weeks.

44. The method of claim 40, wherein the inflammatory disorder is edema.
